(19) United States
(12) Patent Application Publication
(10) Pub. No.: US 2001/0003851 A1
(43) Pub. Date: Jun. 21, 2001

(54) Hair colorant composition containing phytantriol

(76) Inventor: Maurice Raymond Hickling, Pertenhall (GB)

Correspondence Address: Mark E. Waddell, Esq., Bryan Cave LLP, 245 Park Avenue, New York, NY, 10167-0034, US

(21) Appl. No.: 09/734,803

(22) Filed: Dec. 12, 2000

(30) Foreign Application Priority Data
Dec. 16, 1999 (EP) 99125082.0

(51) Int. Cl. A61K 7/13

(52) U.S. Cl. 8/428

(57) ABSTRACT

The present invention discloses hair colorant compositions having improved wash fastness. These compositions contain phytantriol, a dispersing agent, and a hair dye.

### FIELD OF THE INVENTION

The present invention relates to a hair colorant composition containing phytantriol, and to methods of making and using the composition.

### BACKGROUND OF THE INVENTION

Phytantriol is chemically identified as 3,7,11,15-tetramethyl-1,2,3-hexadecanetriol, and is commercially available, e.g., from F. Hoffmann-La Roche AG, Basel, Switzerland.

Vinski et al., U.S. Pat. No. 5,776,443 discloses a hair care composition comprising 0.001-1wt % of phytantriol; 0.001-10 wt % of a silicone compound; and a carrier.

Ribier et al., U.S. Pat. No. 5,834,013 discloses a cosmetic or dermatological composition in the form of an aqueous and stable dispersion of cubic gel particles based on phytantriol and a use thereof for hydrating the skin. The composition essentially includes: (a) from 0.1 to 15% by weight of phytantriol, and (b) from 0.1 to 3% by weight of a dispersing and stabilizing agent, e.g. Tween 20.

### SUMMARY OF THE INVENTION

The documents set forth above do not mention any effect of phytantriol concerning the wash fastness of hair dyes. It has now been found that the addition of phytantriol to a hair colorant composition significantly improves the wash fastness of the hair dye, i.e., significantly improves the color durability of the dye on the hair upon washing.

Accordingly, the present invention relates to the use of phytantriol in a hair colorant composition to improve the wash fastness of dyed hair.

In another aspect, the present invention relates to a hair colorant composition containing phytantriol in an effective amount to improve the wash fastness of hair dyed with a colorant composition.

Another embodiment of the invention is a hair colorant composition containing from about 0.1 to about 5% by weight of phytantriol, from about 0.1% to about 3% by weight of a dispensing agent, and from about 0.1 to about 5% by weight of a hair dye.

A further embodiment of the invention is a hair coloring kit containing a first pack comprising a primary intermediate, a second pack comprising a secondary intermediate, and a container for housing the first and second packs, wherein a phytantriol and dispersing agent composition containing from about 0.1 to about 5% by weight of phytantriol, from about 0.1 to about 3% by weight of a dispersing agent, and from about 0.1 to about 5% by weight of a hair dye is present in at least one of the first and second packs.

Another embodiment of the invention is a method for improving the wash fastness of dyed hair. This process includes applying to the hair a composition containing from about 0.1 to about 5% by weight of phytantriol, from about 0.1 to about 3% by weight of a dispersing agent, and from about 0.1 to about 5% by weight of a hair dye.

Another embodiment of the invention is a kit for coloring hair. The kit has a first pack containing a primary intermediate, a second pack containing a secondary intermediate, together the first and second packs provide from about 0.1 to about 1% of a hair dye, wherein a composition containing from about 0.1 to about 5% by weight of a phytantriol and from about 0.1 to about 3% by weight of a dispersing agent, is present in at least one of the first and second packs; and a container for housing the first and second packs.

### DETAILED DESCRIPTION OF THE INVENTION

More particularly, the present invention is to a hair colorant composition containing:

from about 0.1 to about 5% by weight of phytantriol, preferably from about 0.2 to about 1% by weight of phytantriol;

from about 0.1 to about 3% by weight of a dispersing agent, preferably from about 0.1 to about 1% by weight of the dispersing agent; and

from about 0.1 to about 5% by weight of a hair dye, preferably from about 0.1 to about 1% by weight of the hair dye; wherein the remainder of the composition is essentially a carrier conventionally used in hair colorant compositions, such as an aqueous carrier.

The dispersing agent in the present compositions can be any dispersing agent (surfactant) conventionally used in hair dye compositions such as for example anionic, cationic, nonionic, amphoteric, zwitterionic (betain) surfactants, and mixtures thereof.

Suitable dispersing agents are e.g. selected from:

(1) polyol alkyl or alkenyl ethers or esters,

(2) N-acylated amino acids and derivatives thereof and N-acylated peptides with an alkyl or alkenyl radical, and salts thereof,

(3) alkyl or alkenyl ether or ester sulfates, and derivatives and salts thereof,

(4) polyoxyethylenated alkyl or alkenyl fatty ethers or esters,

(5) polyoxyethylenated alkyl or alkenyl carboxylic acids and salts thereof,

(6) N-alkyl or N-alkenyl betaines,

(7) alkyltrimethylammonium or alkenyltrimethylammonium and salts thereof, and mixtures of the above dispersing agents.

In the compounds listed above, the alkyl and alkenyl radicals preferably have from 8 to 22 carbon atoms.

Preferred dispersing agents are polyol alkyl or alkenyl ethers or esters. Other preferred dispersing agents include sorbitan alkyl or alkenyl esters polyoxyethylenated with at least 20 units of ethylene oxide, such as sorbitan palmitate 20 EO or Polysorbate 40 marketed under the tradename Montanox 40 DF by the company Seppic, and sorbitan laurate 20 EO or Polysorbate 20 marketed under the tradename Tween 20 by the company ICI. Tween 20 is preferred.

Examples of suitable hair dyes used in the present invention include all conventional hair dyes, particularly permanent hair dyes, semipermanent hair dyes, and temporary hair dyes (or hair coloring rinses). Permanent (or oxidative) hair dyes include a component that requires an oxidizing agent for formation of a color (the component being referred to also as a “primary intermediate,” a developer or a precursor), usually a p-phenylenediamine, 2,5-diaminotoluene or 4-aminophenol, and a second component (i.e., a “secondary intermediate” or coupler), e.g., a m-phenylenediamine, 3-aminophenol, 5-amino-2-methylphenol or 1-naphthol, as particularly disclosed e.g., in WO 00/07550.

Semipermanent hair dyes include compounds having a chromophore system that is common in dye chemistry such as nitro, azo, anthraquinone, triphenylmethane and azomethane, which may be used as disperse dyes, cationic (basic) dyes, anionic (acidic) dyes and metal complex dyes. See also Ullmann's Encyclopedia of Industrial Chemistry Vol A 12, p. 584-586 and references cited therein. Of particular interest are e.g. anthraquinone dyes, especially those containing a dialkylaminoalkylamino (DAAA) group. Specific examples include the following compounds available under the trademark ARIANOR® from Warner Jenkinson Ltd.: ARIANOR Straw Yellow, ARIANOR Mahogany, ARIANOR Steel Blue, ARIANOR Madder Red, ARIANOR Ebony and ARIANOR Sienna Brown. Further examples of hair dyes for use in the present invention are those listed in the “Verordnung über kosmetische Mittel, Bundesgesetzblatt (Germany) I, 2410, 29. Aenderung vom 14.6.2000, Teil A, p. 846, which is incorporated by reference as if recited in full herein.

Any other hair dye that is effective in the present compositions may be used. For example, semi-permanent (direct) hair dyes such as those commercially available under the following tradenames may also be used: Irgalan, Cibalan, Vialon, Ortalan and Capracyl dyes. Natural direct dyestuffs, such as Henna, Chamomile, Madder root, Sandalwood or Walnut, likewise may also be used.

The compositions of the present invention may be incorporated into various formulations including, for example, liquids, a gels or mousses. Specific examples of such formulations include a shampoo, a conditioner, a styling mousse, a styling spray, a styling gel, a conditioning styling gel, or a gel mousse. These formulations may, in addition to a hair colorant, contain agents conventionally used in such formulations, such as amphoteric surfactants, thickeners, conditioning agents, sequestrants (to remove heavy metals), preservatives, perfumes and deionized water.

The compositions in accordance with the invention are suitably prepared by dissolving an appropriate amount of phytantriol in an appropriate amount of a dispersing agent, and adding the mixture to an essentially aqueous hair care formulation, such as a solution of a hair dye, a styling mousse, gel or spray, or a shampoo or conditioner. It is not required that the compositions contain any preferred molecular orientation of the phytantriol.

When the hair dye is a permanent (or oxidative) hair dye, the composition according to the invention may be incorporated into a kit of separately packed primary intermediate and secondary intermediate and, optionally, an oxidizing agent. At least one of the separate packs contains phytantriol and a dispersing agent in an amount defined earlier. Examples of oxidizing agents include hydrogen peroxide and organic peroxy compounds such as peracetic acid, which may be used in an amount known to those skilled in the art, e.g., in an amount of from 0.001 to 0.05 moles per 100 g of the composition. Therefore, the invention also relates to a hair coloring kit containing:

from about 0.1 to about 5% by weight of phytantriol, preferably from about 0.2 to about 1% by weight;

from about 0.1 to about 3% by weight of a dispersing agent, preferably from about 0.1 to about 1% by weight; and

from about 0.1 to about 5% by weight of a hair dye, preferably from about 0. 1 to about 1% by weight;

wherein the hair dye consists of separately packed primary and secondary intermediate and the phytantriol and dispersing agent are present in at least one of the separate packs.

The composition according to the invention may be applied to the hair by conventional techniques used in this art. e.g. with a brush, sponge, or other means of contact, such as pouring the composition directly onto the hair until saturated and/or manually massaging or working through the hair.

The following examples are provided to further illustrate the compositions and processes of the present invention. These examples are illustrative only and are not intended to limit the scope of the invention in any way.

### EXAMPLES

### Example 1

Preparation of the hair colorant composition

0.2 g (wt) Phytantriol was dissolved in 0.8 g (wt) dispersing agent (Tween 20) and this mixture was added to 99.0 g of a 0.5% aqueous solution of a hair dye.

### Example 2

Hair Dyeing

The coloring composition of Example 1 was applied to approximately 10 cm long hair swatches for 10 minutes. The hair swatches were then washed with water and allowed to dry in the air. This cycle was repeated 5 times and 10 times, respectively.

### Example 3

Color measurement

Hair colorant compositions containing 0, 0.1 wt %, or 0.2 wt % phytantriol were prepared according to Example 1 and applied to hair swatches according to Example 2.

Hair colors may be defined using the luminance (L) measurement system, according to the colorimetric (L,a,b) coordinate system of the C.I.E (Comite International de 1'Eclairage International Lighting Committee).

In this color measurement system, the color of the hair along three mutually perpendicular axes is measured photometrically and the color differences between the initial sample of hair before washing and the samples after 5 or 10 washes are compared.

The difference between the two samples in the CIELAB space is given by

ΔE=\sqrt{[Δ(Δa)²+(Δb)²+(ΔL)²]}

ΔA is the color difference

Δa is the color difference along the a axis

Δb is the color difference along the b axis

ΔL is the difference in luminance

The following Table shows the results:

| ARIANOR dye | Wt % Phytantriol | ΔE after 10 washings |
|---|---|---|
| A. Straw Yellow | 0 | 16.7 |
| | 0.1 | 15.5 |
| | 0.2 | 12.7 |
| A. Mahogany | 0 | 15.7 |
| | 0.1 | 15.4 |
| | 0.2 | 4.2 |
| A. Steel Blue | 0 | 27.0 |
| | 0.1 | 25.2 |
| | 0.2 | 14.3 |
| A. Madder Red | 0 | 35.8 |
| | 0.1 | 34.4 |
| | 0.2 | 20.7 |
| A. Ebony | 0 | 23.7 |
| | 0.1 | 23.4 |
| | 0.2 | 9.9 |
| A. Sienna Brown | 0 | 17.0 |
| | 0.1 | 16.9 |
| | 0.2 | 9.6 |

The above table clearly shows that the hair dye is more washfast when the hair dyeing treatment has been done using a hair colorant compositions containing phytantriol, especially in an amount of at least 0.2 wt %.

The invention being thus described, it will be obvious that the same may be varied in many ways. Such variations are not to be regarded as a departure from the spirit and scope of the invention and all such modifications are intended to be included within the scope of the following claims.

What is claimed is:

1. A hair colorant composition comprising from about 0.1 to about 5% by weight of phytantriol, from about 0.1% to about 3% by weight of a dispensing agent, and from about 0.1 to about 5% by weight of a hair dye.

2. A hair colorant composition according to claim 1 wherein the phytantriol is present in the composition from about 0.2 to about 1% by weight, the dispersing agent is present in the composition from about 0.1 to about 1% by weight; and the hair dye is present in the composition from 0.1 to about 1% by weight.

3. A composition according to claim 1 wherein the dispersing agent is selected from the group consisting of a polyol alkyl, an alkenyl ether, and an ester.

4. A composition according to claim 3 wherein the dispersing agent is TWEEN 20.

5. A composition according to claim 1 wherein the hair dye is a permanent dye.

6. A composition according to claim 1 wherein the hair dye is a semi-permanent dye.

7. A composition according to claim 6 wherein the dye is an anthraquinone dye.

8. A hair colorant composition according to claim 7 wherein the anthraquinone dye is selected from the group consisting of ARIANOR Straw Yellow, ARIANOR Mahogany, ARIANOR Steel Blue, ARIANOR Madder Red, ARIANOR Ebony, and ARIANOR Sienna Brown.

9. A kit for coloring hair comprising:
(a) a first pack comprising a primary intermediate;
(b) a second pack comprising a secondary intermediate, together the first and second packs provide from about 0.1 to about 1% of a hair dye, wherein a composition comprising from about 0.1 to about 5% by weight of a phytantriol and from about 0.1 to about 3% by weight of a dispersing agent, is present in at least one of the first and second packs; and
(c) a container for housing the first and second packs.

10. A kit according to claim 9 wherein the phytantriol is present in the composition from about 0.2 to about 1% by weight, the dispersing agent is present in the composition from about 0.1 to about 1% by weight, and the hair dye is present in the composition from 0.1 to about 1% by weight.

11. A method for improving the wash fastness of dyed hair comprising:
(a) applying to the hair a composition comprising from about 0.1 to about 5% by weight of phytantriol, from about 0.1 to about 3% by weight of a dispersing agent, and from about 0.1 to about 5% by weight of a hair dye.

12. A hair coloring composition formed by the process comprising:
(a) dissolving from about 0.1% to about 5% by weight of a phytantriol in a about 0.1% to about 1% of a dispersing agent; and
(b) mixing the composition of (a) with about 0.1% to about 5% by weight of a hair dye.
